(19) United States
(12) Patent Application Publication
(10) Pub. No.: US 2017/0068791 A1
(43) Pub. Date: Mar. 9, 2017

(54) PRECLINICAL CARE SYSTEM

(71) Applicant: WEINMANN EMERGENCY MEDICAL TECHNOLOGY GMBH + CO. KG, Hamburg (DE)

(72) Inventors: Frank Hermann, Barmstedt (DE); Tobias Kalwa, Hamburg (DE); Felix Klockner, Hamburg (DE); Ulrich Palm, Norderstedt (DE); Norman Scotti, Hamburg (DE)

(21) Appl. No.: 15/309,340

(22) PCT Filed: Apr. 24, 2015
(86) PCT No.: PCT/DE2015/000212

§ 371 Date: Nov. 7, 2016

(30) Foreign Application Priority Data
May 7, 2014 (DE) 10 2014 006 825.0

(51) Int. Cl. G06F 19/00; A61M 16/00; A61B 5/00; A61N 1/39

(52) U.S. Cl. CPC G06F 19/345; A61N 1/3937; A61N 1/3987; A61M 16/00; A61B 5/746; A61B 2505/01; A61B 5/0402

(57) ABSTRACT

A system having preclinical emergency care modules which produce a complex condition dependent control system that is as integral as possible in combination with a human emergency worker. At the system level, an intelligent decision-making system is provided which leads to measures that are optimized for the situation with and without the emergency worker. The modules exhibit a different behavior depending on the situation and interaction. In the process, the emergency worker can be utilized as an additional sensor/actuator module. Based on all obtained sensor data, which is weighted differently, decision-making support is proposed to the emergency worker, or the system makes decisions automatically. The protected communication of the modules is of particular importance for this purpose.

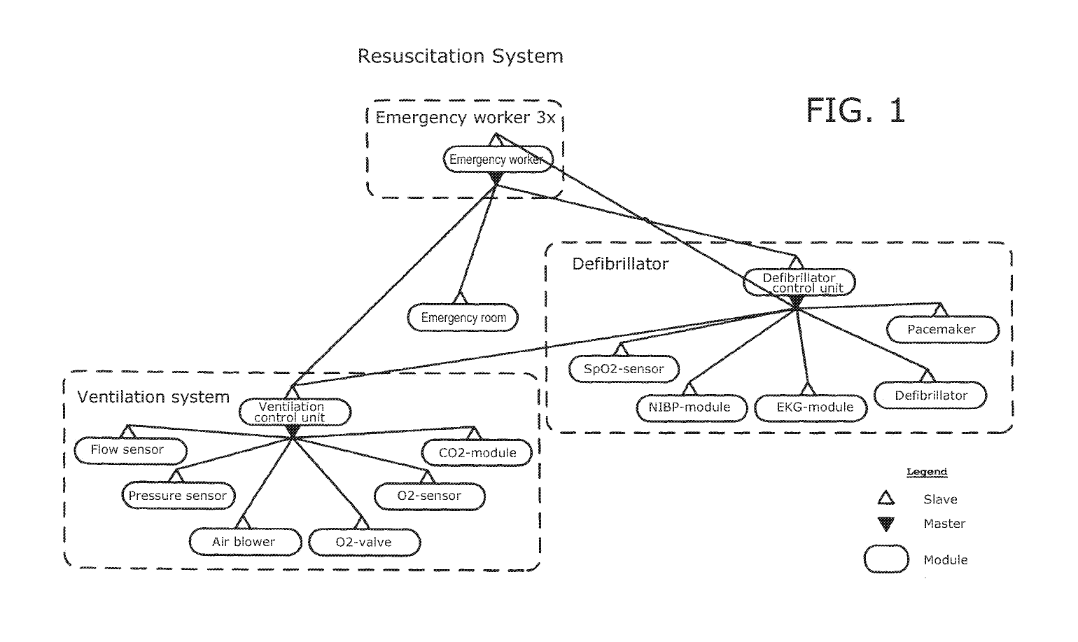

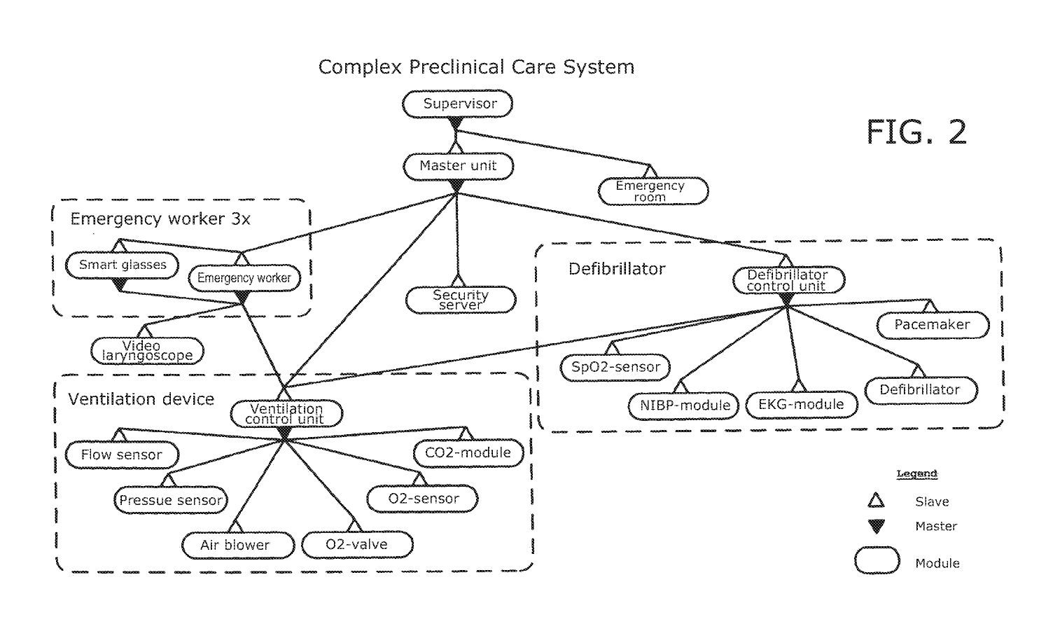

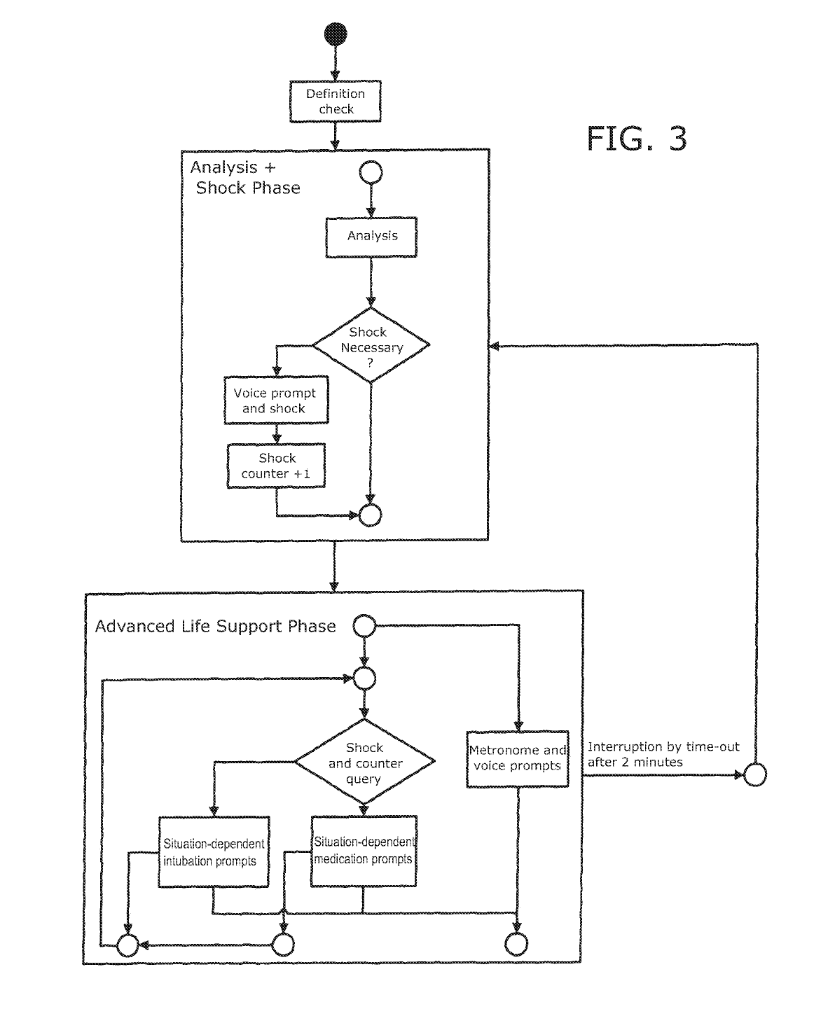

The invention pertains to a system consisting of preclinical emergency care modules, through the combination of which a complex, highly integrated, situation-dependent control system is obtained in conjunction with a human emergency worker.

For “basic life support”, US Patent No. 2006 111,749 A1 describes a voice-guided system which provides an emergency worker with support in a cardiopulmonary emergency situation. A defibrillator and a ventilator with typical sensors are on hand, which control the emergency care with the help of flow charts.

In U.S. Pat. No. 6,327,497 B1, a method and a device for controlling a defibrillator are described, in which a voltage generator is used to apply an electroshock voltage to electrodes. In addition, oxygen is conducted from a compressed gas cylinder to a delivery element. The delivery of the oxygen is controlled by a metering element for performing oxygenation.

EP 1 369 144 B1 describes a device for defibrillation and a ventilation device with a control unit for the synchronized performance of successive inspiration and expiration phases, for the performance of a fibrillation analysis, and for a subsequent defibrillation upon detection of a fibrillation state.

EP 1 264 615 B1 describes a therapy device with a ventilation unit, a defibrillation unit, a fibrillation detector, and a control unit, which is connected to the ventilation unit and to the control unit and is configured, first, to actuate the ventilation unit to ventilate the patient. The device described here also consists of a therapy device configured to interrupt the ventilation for the purpose of VF detection by the fibrillation detector and, if VF is detected, to actuate the defibrillation unit.

Common to all of the previously published solutions is the combination of various modules, which are connected to each other. The emergency worker often participates in the process on the basis of acoustic, optical, or text outputs. The processes proceed through one or more fixed sets of instructions. A decision circuit is resident on the module level, but not on the system level.

The goal of the present invention is to realize a device of the type described above in such a way that an intelligent decision system is present on the system level, which leads to measures optimized for the situation with or without the participation of the emergency worker.

This is to be realized in such a way that the modules demonstrate different types of behavior depending on the situation and the interactions between them. The emergency worker can function as an additional sensor-actuator module. As a function of all of the acquired sensor data, which are weighted in different ways, the system supplies the emergency worker with decision aids or makes its own decision independently. To make this possible, the secure communication of the modules among themselves is especially important. Communication with the “emergency worker” module takes place acoustically (alarms and/or prompts), optically, tactilely, or by the use of any other type of human sense perception. The system also receives the actions or feedback from the emergency worker by way of acoustic signals (speech recognition), gestures, or input sensors.

This goal is achieved according to the invention in that the connectability via data transmission means makes it possible for address, time, and status signals to be exchanged, wherein the system device comprises at least one sensor device for acquiring status signals, a time-measuring device, and a data storage unit; the goal also being achieved in that the modules are configured to link a status signal of the associated sensor device with a time signal of the time-measuring device, and in that at least one module of the system device transmits these data together with an address code over at least one interface by the use of a data transmission protocol.

According to the invention, the control means automatically and dynamically generates, changes, and/or supplements assignment data as a reaction to a manual actuation, a specific electronic actuation, a detected transmission problem of the data transmission means, and/or an activation or deactivation of a module, which data are then stored in electronic storage means.

One module is configured to generate, send, and/or receive a confirmation signal for an action performed by another module.

According to the invention, a first functional unit with first digital address data can be actuated and identified via the data transmission means. An associated actuator unit of the first functional unit can thus be actuated by means of stored primary operating data, wherein the primary operating data are stored in the area of the first functional unit and can be called up as needed, or the actuator unit can be actuated on the basis of secondary operating data received and/or receivable over the data transmission means. Secondary operating data in that case always have higher priority.

According to the invention, at least one of the functional units is configured so that it can be equipped, upgraded, and retrofitted in modular fashion with modules specifically configured for hard-wired and/or wireless data transmission over the data transmission means.

According to the invention, the system device comprises a hard-wired communications device for connecting at least two functional units and/or a wireless communications device for connecting at least two functional units.

A master-slave concept has proven favorable for connecting the modules. A master has control over at least one slave. The slave acts independently on its module level (makes measurements and/or executes controls in its own sphere of activity) and transmits its values to the master. The master, for its own part, can influence the slave on the basis of other data collected in the system, just as the slave's data can influence the master.

A master can itself be a slave when subordinate to a higher-priority master, so that a hierarchical structure is obtained. The emergency worker can be both a master and a slave. There will usually be one maximum-priority system master, which is formed by any desired “human” or technical module.

During the performance of care measures, a change from one system master to another is conceivable and in many cases advisable. Thus the technical system could act as master and thus lighten the worker's burden. If the patient's condition changes, however, the worker can resume the function of master. Or in the case of a resuscitation, the defibrillator starts out as the master, and if the heart's activity is restored successfully, it passes on the master function to the ventilator, which controls respiration as a function of the CO₂ and SpO₂ values and receives the ECG data for monitoring purposes.

For the secure communication of the modules with each other, data transmission with checksums or the like and feedback are required. Also required is the unique identification of the data and the associated assignment of the data to the transmitting modules. Important for all of the communications data is the acquisition time. In the event that the communication breaks down, repeated attempts will be made to establish a connection, unless the user has actively interrupted it. In the meantime, the modules work autonomously or switch over to a passive mode.

Communication with the emergency worker is especially important. Measures for the unique identification of the user (fingerprint reader, RFID armband, voice recognition, iris scan, etc.) are helpful here or perhaps even necessary on legal grounds. Acoustic communication from and to the worker is especially important, so that the emergency worker has his hands free for his supportive measures. Measures performed by the emergency worker should also be receipted.

A special solution could be a pair of smart glasses. All useful information is displayed on them to the emergency worker. Video data like those from a video laryngoscope can be displayed. An iris scan can be used for identification. Eye movements and acoustic signals can be used for communication.

The management of alarm signals is especially important. Alarms which are meaningful on the module level can have a completely different priority and meaning on the system level. The system master might influence the alarm output on the module level and/or prioritize it. In the case of a resuscitation, for example, certain alarms given by the ventilator, for example, would be suppressed during chest compression until controlled breathing resumes.

A resuscitation device consists essentially of, for example, the modules “ventilator” and “defibrillator”. The modules communicate over a cable or by radio, thus exchanging address, time, and status signals as well as sensor signals. The ventilator, as a slave element, comprises pressure and flow sensors, which record the pressure and flow signals of the breathing gas and report these to the ventilation master. The ventilation uses this information to control the actuators, which operate as slaves.

The defibrillator comprises two electrodes for detecting status signals of the heart's activity (ECG). The ECG module works as a slave under the control unit (master) of the defibrillator. The defibrillator and the ventilator also have time measuring devices. These serve to generate a system time, which—in the form of a time stamp—is linked with the sensor signals (pressure, flow, ECG).

The defibrillator and the ventilator also have individual address codes, by means of which the devices can be identified. The defibrillator and the ventilator also comprise control units and are configured to transmit, via a data transmission protocol, the associated sensor data or control signals to the associated other device.

The data of the sensors are typically linked with the associated address code and the associated time stamp of the associated time measuring device and then either stored and/or transmitted over the data transmission means to the associated other device. Often these data are transmitted together with control signals, because, in certain cases, the sensor data represent events which cause the ventilator or the defibrillator to perform an action. The transmission of the control signals proceeds preferably by way of the data transmission means.

On the basis of the acquired data, the system (master) decides in a situation-dependent manner what measures must be carried out by the emergency worker (slave). So that he can perform these measures, the emergency worker will then be instructed visually and acoustically by the voice prompts and text messages or pictograms. The performance of these measures is confirmed manually by the emergency worker, so that the system can initiate the further steps. The confirmed manual measures are stored and added to the total deployment data, including time stamp. These measures which can be carried out manually by the user include:

performing chest compression at the right time, at the optimum frequency and depth, in the optimum position, and with sufficient load release;

analyzing cardiac rhythm and defibrillation;

preparing for and implanting intravenous/intraosseous access;

preparing for and performing intubation;

preparing and administering medications for resuscitation;

administering therapeutic hypothermia; and

adjusting the ventilation parameters.

In the case of ongoing ECG recording and analysis, the defibrillator generates a control signal, which is transmitted to the ventilator, whereupon the ventilator stops generating breaths upon termination of the first expiration following the control signal. The defibrillator takes over the function of system master.

This is necessary in certain cases, because the strokes of the ventilator can interfere with the ECG analysis. While the defibrillator is preparing to administer a shock and even while it is charging as well as during the shock administration itself, the ventilator strokes continue to be suppressed. The emergency worker usually triggers the shock by pushing a button after being instructed to do so by the system master (defibrillator). The emergency worker serves here as an actuator, although he still retains a certain freedom to make decisions.

The sensors of the ventilator record the patient's respiratory drive, whereupon the ventilator generates the feedback “the patient is breathing” to the defibrillator. In addition or as an alternative, the ventilator generates the message “the patient is breathing” on the display or by means of other signal devices.

All of the sensor and actuator signals can be acquired and processed on the basis of the complex communications within the system.

In the following, various sensor and/or actuator signals are listed by way of example:
(a) device data:
   maintenance dates
   location (GPS)
   hours of operation
   battery status
   function controls
   chronology (power on, power off)
(b) monitor module data:
   ECG (3, 6 or 12-channel)
   CPR feedback data
   intracranial pressure measurement
   NIBP [Noninvasive Blood Pressure Amplifier]
   SpO₂
   FiO₂
   etCO₂
   temperature
   alarms, alarm limits, events
   chest compressions
   volume
   pressure (PEEP [Positive End-Expiratory Pressure], plateau pressure, peak pressure)
(c) ventilator module data:
   ventilation mode
   respiratory values (tidal volume, Pᵢₙₛₚ, respiratory rate, MV [minute volume], FiO₂, peak, PEEP, CO₂)
   flow O₂ inhalation
(d) defibrillation module data:
   defibrillation
   cardioversion
   automatic ECG analysis
   pacemaker

Manually performed measures (e.g., administration of medications) are carried out preferably with the possibility of free text.

For example, the following data can be added:

patient data (medical insurance card, health card),

deployment data (personnel, operator, vehicle, deployment no., deployment times),

CPR thumper data.

Protocols can be defined and documented for the integration of data from other devices.

Data can be printed out in a protocol at the deployment site, so that they can be made available to the physician providing further care.

Data can be transmitted from the deployment site in real time to supervisors for telemedical consultation.

Data can be transmitted from the deployment site to central servers for quality assurance.

Data can be fed into standard clinical documentation systems.

The alarms and prompts of both devices are transmitted/displayed at the time they occur (i.e., no prioritization of one device over the other). Meaningless alarms are therefore not triggered (the patient alarms “disconnection” and “stenosis”, which remain current during chest compression in the CPR phase are not active during the analysis phase or shock preparation phase).

If a shock for which preparation has been made is not administered, respiration is started again after the internal discharge has been completed. The “mask/tube” button on the ventilator has the function of limiting the respiratory pressure to 20 or 45 hPa. The choice between “CPR in a 30:2 rhythm” and “pushthrough” is made on the defibrillator in both AED [Automated External Defibrillator] and manual modes. The stenosis alarm is triggered when a ventilation stroke and chest compression occur simultaneously during push through.

Alarm outputs can be reduced to a minimum by intelligent processes. This means that not only the alarm condition but also additional active parameters/processes are checked before an alarm is triggered.

The alarm conditions are to be ignored during resuscitation, because it is known that the patient is in a must-resuscitate state, and the CPR measures are the only ones which can counter the conditions on which the alarm is based.

No asystole or VF/VT alarm is triggered if a pulse curve is present.

It should be possible to assign visual alarm outputs easily and quickly to the module originating the alarm.

Acoustic alarm outputs must be module-specific, so that they can be assigned to the module triggering the alarm.

The loudness of the alarm output should be adjustable and should adapt itself automatically to the ambient noise level.

It should be possible to adapt the adjustable alarm limits to the patient-specific conditions with minimal effort (e.g., by means of auto alarm limits).

The commands from the defibrillator (master) are transmitted to the ventilator (slave).

When a connection is established, the master always receives a feedback message in response to its commands.

The following parameters are taken into consideration when a command is given:

The interface is configured for unsynchronized communications with the auditing computer.

The firmware of the ventilator can be upgraded in such a way that speech can be turned off, alarms suppressed or terminated, respiration stopped or started, or the stenosis alarm limit changed.

The ventilator can send back its settings (frequency), the respiratory phase, or the type of alarm which is currently in effect as parameters. The airways pressure can be transmitted and displayed as monitoring data.

During the communication between the defibrillator and the ventilator, the defibrillator assumes the role of master. The ventilator works as a slave and reacts only to commands received from the defibrillator.

The defibrillator begins the communication by sending an appropriate command to the ventilator. After sending a message to the user, the ventilator then turns off its speech output and transmits the appropriate reply to the defibrillator.

If the communication is interrupted while the ventilation is stopped, the ventilator resumes the ventilation, because it has not received any new command.

By issuing the “start” command, the defibrillator can cancel the interruption of the ventilation at any time. In this case the ventilator begins the ventilation either immediately or after termination of any inspiration/expiration phase which may be present.

The defibrillator monitors the number of ventilation strokes and keeps track of whether or not the ventilator is remains ready to operate (demand mode selected by the user).

The defibrillator can end the communication by transmitting the appropriate command. In this case, the ventilator stops sending any data, continues the ventilation autonomously, and confirms the termination of the communication by echoing the command.

If, as the result of a transmission error, the ventilator does not understand the defibrillator's command, it sends a command to repeat the transmission. The defibrillator then repeats the command. If, after several repetitions, there has been no successful confirmation of the command by the ventilator, the defibrillator breaks off the communication, and both devices continue to operate autonomously.

After communication has been broken off as the result of a communications error, the defibrillator continues to request the data as long as it is not receiving an answer from the ventilator. If the defibrillator receives a defective answer from the ventilator, it repeats the command up to two times and then again after 3 seconds. If the expected data package is then received, communications are resumed again.

In the following, a complex care system is to be described on the basis of an accident situation by way of example. The emergency team is called to a traffic accident involving a pedestrian. Having arrived at the deployment site, the team attempts to talk to the injured person and to reconstruct the course of the accident. One of the three emergency workers describes the condition of the patient and the situation verbally to the system. The system stores these data.

A higher-level on-site supervisor of the action team, which carries with him a control and monitoring master unit for monitoring purposes, receives the medical history data, which are then displayed on the master unit. The emergency workers turn on their smart glasses. The data on the master unit are sent to the smart glasses of the individual action team members and displayed there. During the deployment, the supervisor's master unit functions as the master and treats the smart glasses and care system such as a defibrillator and ventilator as slaves.

By means of iris scans, the smart glasses identify the emergency workers and know their skills immediately. The patient data are then displayed on the smart glasses of the action team members. The emergency workers begin with the monitoring of the patient. The emergency workers connect sensors to the patient to measure the vital parameters such as oxygen saturation, blood pressure, and pulse. An ECG is also connected to the patient.

The data are transmitted via the on-site supervisor's master unit to the individual smart glasses of the emergency workers and displayed there. Because the state of the patient is becoming worse, the system transmits warnings individually to the supervisor and to the emergency workers.

The emergency workers notice that the patient is losing consciousness; his breathing is becoming weaker; and clearly audible rales are heard as he is breathing. The recorded vital parameters are continuously transmitted to the master unit of the supervisor. On the basis of the patient's situation, the supervisor establishes contact with an expert at the nearest emergency room. The patient's data are transmitted in real time to the ER expert.

Because of the deteriorating blood pressure of the patient, additional measures are recommended by the expert.

Because of the suspicion of a concussion caused by the traffic accident, an intracranial pressure measurement is conducted on the skull of the patient. The abdominal space of the patient is also subjected to an ultrasound examination. The images from the two systems are transmitted wirelessly to the smart glasses of the emergency workers and supervisor.

Internal bleeding is discovered. The patient's condition continues to worsen until the point that resuscitation becomes necessary. The emergency workers begin the resuscitation immediately and decide to intubate. A video laryngoscope is used to display a video image of the patient's throat on the smart glasses. The video image makes it possible for the patient to be intubated despite a difficult-to-access position. The suction pump is activated by voice commands. As soon as the tube is seated, the ventilator is activated by speech commands.

The respiratory status is monitored, and the data are displayed on the master unit and on the smart glasses of the emergency workers. All actions are registered by a camera in the smart glasses of the emergency workers and stored in the document memory of the master unit. These are transmitted to a central backup server for later billing for the deployment and for quality assurance and for improving the performance of the participating team members.

During the resuscitation, the system recognizes what measures the emergency workers have already taken and then, by means of an algorithm-based evaluation, gives recommendations for additional therapeutic alternatives. The workers decide to transport the patient as quickly as possible to the nearest emergency and trauma clinic. Because the patient continues to be in need of resuscitation, the action team members decide to use an automated resuscitation device for the trip to the hospital.

The care system assumes wireless control of the resuscitation device, so that an optimal sequence of ventilation and chest compression can take place. At this moment, the defibrillator of the care system takes over the role of master to control the ventilator and the resuscitation device. The care system recognizes the state of the patient and during the deployment gives voice prompts with additional action recommendations. Because the on-site supervisor has already transmitted the data to the expert in the hospital with the help of telemedical support, the hospital can make optimal preparations for the arrival of the patient.

1-20. (canceled)

21. A device for preclinical emergency care, comprising a system having at least two networked modules, which make a situation-dependent course of action possible through the networking.

22. The device according to claim 20, wherein the modules contain sensors, actuators, input and output elements, and/or open-loop/closed-loop control elements.

23. The device according to claim 20, wherein an emergency worker is used as a module in the system.

24. The device according to claim 20, wherein the network uses wired, wireless, optical, acoustic, magnetic, and/or tactile transmission techniques.

25. The device according to claim 20, wherein the network makes possible unique module/data identification with time stamp and secure communication.

26. The device according to claim 20, wherein, if communication is interrupted, lower-level and higher-level modules continue to work autonomously.

27. The device according to claim 20, wherein one module is a defibrillator and one module is a ventilator.

28. The device according to claim 27, wherein intelligent, situation-dependent voice prompts support an emergency worker.

29. The device according to claim 20, wherein, video laryngoscopes, CPR feedback systems, remote-control modules, ultrasound systems, cranial pressure measurement systems, smart glasses, documentation systems, and/or other sensor, actuator, and/or HMI modules are integratable into the system.

30. A device for preclinical emergency care, comprising a system with modules that have an organizational structure for controlling a course of events.

31. The device according to claim 30, wherein the organizational structure contains master-slave principles.

32. The device according to claim 30, wherein master-slave lower-level structures serve as slaves for higher-level structures in the organizational structure.

33. The device according to claim 30, wherein there is a structure with a system master.

34. The device according to claim 30, comprising several parallel, equal structures and/or system masters.

35. The device according to claim 33, wherein the system master is a human being or a device.

36. The device according to claim 35, wherein the system master changes during care procedures.

37. The device according to claim 30, wherein modules are addable to and removable from the system during the course of events.

38. The device according to claim 30, wherein system masters monitor alarm outputs and control them as a function of a situation.

39. A device for preclinical emergency care, comprising a system that optimizes a therapy process in a user-supported and/or autonomous manner.

40. The device according to claim 39, wherein the system exercises control as a function of a situation in an open-loop or closed-loop manner.
